(19) United States
(12) Patent Application Publication
(10) Pub. No.: US 2024/0101122 A1
(43) Pub. Date: Mar. 28, 2024

(54) DRIVER PRE-ABNORMAL DETECTION APPARATUS, CIRCUIT AND COMPUTER PROGRAM THEREFOR

(71) Applicant: Mazda Motor Corporation, Hiroshima (JP)

(72) Inventors: Akiko FUKUI, Hiroshima (JP); Koji IWASE, Hiroshima (JP); Yohei IWASHITA, Hiroshima (JP)

(73) Assignee: Mazda Motor Corporation, Hiroshima (JP)

(21) Appl. No.: 18/126,506

(22) Filed: Mar. 27, 2023

(30) Foreign Application Priority Data
Sep. 28, 2022 (JP) 2022-154856

(51) Int. Cl. B60W 40/08; B60W 40/02; B60W 50/14; G06V 20/59

(52) U.S. Cl. CPC B60W 40/08; B60W 40/02; B60W 50/14; G06V 20/597; B60W 2540/225; B60W 2540/229

(57) ABSTRACT

A driver pre-abnormal detection apparatus includes: sensors to acquire travel environment information of a vehicle; an in-vehicle camera that detects the driver's sightline; and a controller configured to detect a driver's pre-abnormal state based on the travel environment information and the driver's sightline. The controller identifies a visual recognition required point to be visually recognized by the driver based on the travel environment information, determines whether the driver has visually recognized the visual recognition required point based on the driver's sightline, and detects the driver's pre-abnormal state based on a fact that the driver has not visually recognized the visual recognition required point.

### CROSS-REFERENCE TO RELATED APPLICATION

The present application claims priority to Japanese Patent Application No. 2022-154856 filed in the Japanese Patent Office on Sep. 28, 2022, the entire contents of which being incorporated herein by reference.

### TECHNICAL FIELD

Embodiments relate to a driver abnormality sign detection apparatus for detecting an abnormality sign state of a driver during driving of a vehicle.

### BACKGROUND ART

Conventionally, a driver state detection apparatus that detects abnormality of a driver of a vehicle has been proposed (for example, see Patent document 1). In the apparatus disclosed in Patent document 1, an amplitude and a frequency of the vehicle driver's saccade (jumping eye motion of the driver to intentionally move his/her sightline) are detected, and such an attention level is detected that is increased as the number of caution points to be checked by the driver during travel is increased in vehicle outside environment, so as to detect the abnormality of the driver based on the attention level and the amplitude and the frequency of the driver's saccade.

### PRIOR ART DOCUMENTS

### Patent Documents

[Patent document 1] JP-A-2021-077136

### SUMMARY

### Problems to be Solved

However, in a state where the driver's attention function is slightly declined due to a mild disease, aging, or the like but the driver can still drive, i.e., an abnormality sign state prior to an abnormal state where the driver has difficulty in driving, no obvious difference appears to the amplitude and the frequency of the driver's saccade in comparison with a state with no abnormality. Thus, the abnormality sign state cannot be detected by the related art as described above.

Embodiments are directed to solving this and other problems and therefore has a purpose of providing a driver abnormality sign detection apparatus capable of detecting an abnormality sign state of a driver at a stage sufficiently earlier than onset of an abnormal state where the driver has difficulty in driving, i.e., to detect a pre-abnormal state between the normal state and the abnormal state. In other words, the pre-abnormal state is a state in which a driver may be able to drive safely, but may benefit from support to drive safely, as opposed to normal state in which a driver can drive safely and an abnormal state in which a driver cannot drive safely.

### Means for Solving the Problems

In order to solve the above-described and other problems, embodiments are directed to a driver abnormality sign detection apparatus that detects an abnormality sign state of a driver who drives a vehicle, and includes: a travel environment information acquisition device that acquires travel environment information of the vehicle; a sightline detector that detects the driver's sightline; and a controller configured to detect the driver's abnormality sign state based on the travel environment information and the driver's sightline. The controller is configured to: identify a visual recognition required point to be visually recognized by the driver based on the travel environment information; determine whether the driver has visually recognized the visual recognition required point based on the driver's sightline; and detect the driver's abnormality sign state based on a fact that the driver has not visually recognized the visual recognition required point.

Accordingly, the controller identifies the visual recognition required point to be visually recognized by the driver based on the travel environment information, determines whether the driver has visually recognized the visual recognition required point based on the driver's sightline, and detects the driver's abnormality sign state based on the fact that the driver has not visually recognized the visual recognition required point. Thus, the abnormality sign state may be detected by using a fact that the driver has not been able to visually recognize the visual recognition required point due to a decline in a distributive attention function or a reduction in an effective visual field at an initial stage of a decline in the driver's attention function. In this way, the driver's abnormality sign state at a stage, at which no obvious difference appears to the amplitude and the frequency of the driver's saccade and which is sufficiently earlier than onset of the abnormal state where the driver has difficulty in driving, for example.

The controller may be configured to: determine that the driver has visually recognized the visual recognition required point in the case where the driver's sightline is directed within a specified range including the visual recognition required point; and detect the driver's abnormality sign state based on the number of the visual recognition required point not visually recognized by the driver.

Accordingly, the driver's abnormality sign state is detected based on the number of the visual recognition required point to which the driver's sightline is not directed. Thus, when the driver can no longer visually recognize the plural visual recognition required points at the same time due to the decline in the distributive attention function and starts missing the visual recognition required points at the initial stage of the decline in the attention function, the abnormality sign state can be detected based on the severity of missing of the visual recognition required point. In this way, the driver's abnormality sign state may be appropriately detected at the stage sufficiently earlier than the onset of the abnormal state where the driver has difficulty in driving.

The controller may be configured to detect the driver's abnormality sign state in the case where a ratio of the number of the visual recognition required point not visually recognized by the driver to the total number of the visual recognition required points is equal to or larger than a specified value.

Accordingly, when the driver can no longer visually recognize the plural visual recognition required points at the same time due to the decline in the distributive attention function and the missing ratio of the visual recognition required point becomes equal to or higher than the specified value at the initial stage of the decline in the attention function, the abnormality sign state can be detected. In this way, the driver's abnormality sign state may be appropriately detected at the stage sufficiently earlier than the onset of the abnormal state where the driver has difficulty in driving.

The controller may be configured to: estimate a size of an effective visual field that can be visually recognized by the driver based on a distance between the driver's sightline and the visual recognition required point not visually recognized by the driver; and detect the driver's abnormality sign state in the case where the estimated effective visual field size is smaller than the specified value.

Accordingly, when the effective visual field is narrowed and the driver starts missing the visual recognition required point at the initial stage of the decline in the attention function, the abnormality sign state can be detected by estimating the degree of the narrowness of the effective visual field. In this way, the driver's abnormality sign state may be appropriately detected at the stage sufficiently earlier than the onset of the abnormal state where the driver has difficulty in driving.

The driver abnormality sign detection apparatus may further include an information output device that outputs information to the driver. The controller is configured to cause the information output device to output information used to guide the driver's sightline to the visual recognition required point in the case where the driver's abnormality sign state is detected.

Accordingly, in the case where the driver's abnormality sign state is detected, the driver's sightline is guided to the visual recognition required point. Therefore, driver assistance may be appropriately provided to the driver who starts missing the visual recognition required point at the initial stage of the decline in the attention function.

### Advantages

According to the driver abnormality sign detection apparatus according to one or more embodiments, the driver's abnormality sign state can be detected at the stage sufficiently earlier than the abnormal state where the driver has difficulty in driving.

### BRIEF DESCRIPTION OF THE DRAWINGS

FIG. 1 is an explanatory view of a vehicle on which a driver abnormality sign detection apparatus according to an embodiment is mounted.

FIG. 2 is a block diagram of the driver abnormality sign detection apparatus according to the embodiment.

FIG. 3 is a control block diagram of abnormality sign detection according to the embodiment.

FIG. 4 is a table exemplifying information stored in a visual recognition required point database according to the embodiment.

FIG. 5 is a flowchart of detection processing of an abnormality sign state according to the embodiment.

### DETAILED DESCRIPTION

A description will hereinafter be made on a driver abnormality sign detection apparatus according to an embodiment with reference to the accompanying drawings.

[System Configuration]

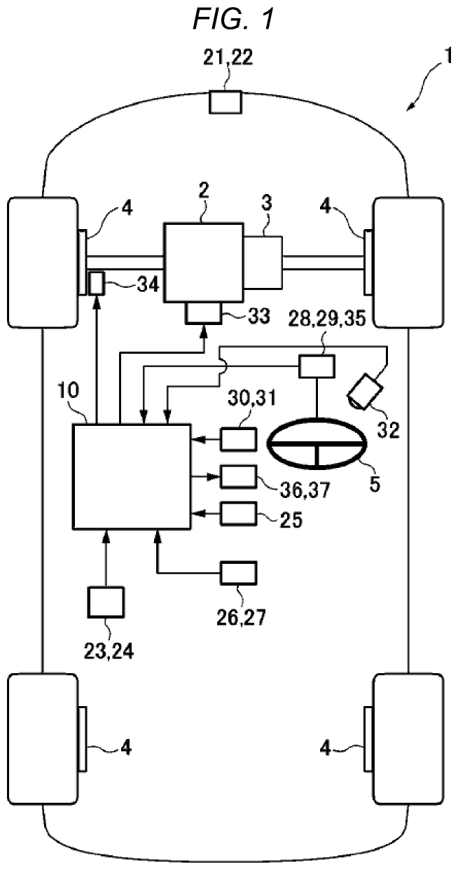

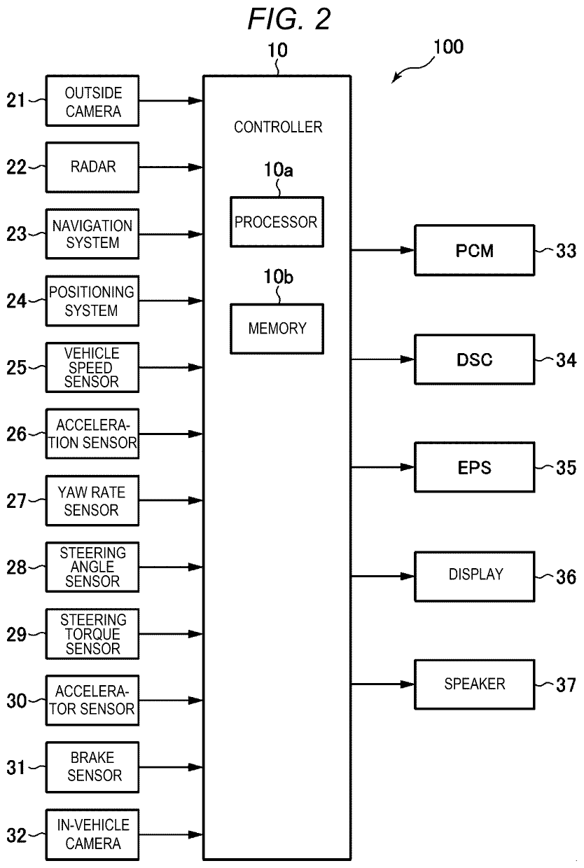

First, a description will be made on a configuration of the driver abnormality sign detection apparatus according to this embodiment with reference to FIG. 1 and FIG. 2. FIG. 1 is an explanatory view of a vehicle on which the driver abnormality sign detection apparatus is mounted, and FIG. 2 is a block diagram of the driver abnormality sign detection apparatus.

A vehicle 1 according to this embodiment includes: drive power sources 2 such as an engine and an electric motor that output drive power; a transmission 3 that transmits the drive power output from the drive power source 2 to drive wheels; a brake 4 that applies a braking force to the vehicle 1; and a steering device 5 for steering the vehicle 1.

A driver abnormality sign detection apparatus 100 is configured to detect an abnormality sign state, i.e., a pre-abnormal state, of a driver of the vehicle 1 and to execute control of the vehicle 1 and driving assistance control when necessary. The pre-abnormal state is a state in which a driver may be able to drive safely, but may benefit from support to drive safely, as opposed to normal state in which a driver can drive safely and an abnormal state in which a driver cannot drive safely. As illustrated in FIG. 2, the driver abnormality sign detection apparatus 100 has a controller 10, plural sensors, plural control systems, and plural information output devices.

More specifically, the plural sensors include an outside camera 21 and a radar 22 for acquiring travel environment information of the vehicle 1 as well as a navigation system 23 and a positioning system 24 for detecting a position of the vehicle 1. The plural sensors also include a vehicle speed sensor 25, an acceleration sensor 26, a yaw rate sensor 27, a steering angle sensor 28, a steering torque sensor 29, an acceleration sensor 30, and a brake sensor 31 for detecting behavior of the vehicle 1 and the driver's driving operation. The plural sensors further include an in-vehicle camera 32 for detecting the driver's sightline. The plural control systems include: a powertrain control module (PCM) 33 that controls the drive power source 2 and the transmission 3; a dynamic stability control system (DSC) 34 that controls the drive power source 2 and the brake 4; and an electric power steering system (EPS) 35 that controls the steering device 5. The plural information output devices include a display 36 that outputs image information and a speaker 37 that outputs voice information.

In addition, other sensors may include a peripheral sonar that measures a distance and a position of a peripheral structure relative to the vehicle 1, a corner radar that measures approach of the peripheral structure to four corner sections of the vehicle 1, and various sensors (a heart rate sensor, an electrocardiogram sensor, a steering wheel grip force sensor, and the like) that detect the driver's state.

The controller 10 executes various arithmetic operations based on signals received from the plural sensors, transmits, to the PCM 33, the DSC 34, and the EPS 35, a control signal for appropriately actuating the drive power source 2, the transmission 3, the brake 4, and the steering device 5, and transmits, to the display 36 and the speaker 37, a control signal for outputting desired information. The controller 10 is a computer that includes one or more processors 10a (typically, a CPU), memory 10b (ROM, RAM, and the like, e.g., a non-transitory storage device) that stores various programs and data, an input/output device, and the like. As used herein ‘computer’ refers to circuitry that may be configured via the execution of computer readable instructions, and the circuitry may include one or more local processors 10a (e.g., CPU's), and/or one or more remote processors, such as a cloud computing resource, or any combination thereof.

The outside camera 21 captures an image, e.g., a visible image, an infrared image, or the like, around the vehicle 1 and outputs image data. The controller 10 identifies an object (a preceding vehicle, a parked vehicle, a pedestrian, a travel road, a lane marking (a lane divider, a white line, or a yellow line), a traffic signal, a traffic sign, a stop line, an intersection, an obstacle, or the like) based on the image data received from the outside camera 21. The outside camera 21 corresponds to an example of the “travel environment information acquisition device” in the disclosure.

The radar 22 measures a position and a speed of the object (particularly, the preceding vehicle, the parked vehicle, the pedestrian, a dropped object on the travel road, or the like). For example, a millimeter-wave radar can be used as the radar 22. The radar 22 transmits a radio wave in an advancing direction of the vehicle 1, and receives a reflected wave generated when the object reflects the transmitted wave. Then, based on the transmitted wave and the received wave, the radar 22 measures a distance between the vehicle 1 and the object (for example, an inter-vehicular distance) and a relative speed of the object to the vehicle 1. In this embodiment, instead of the radar 22, a laser radar, an ultrasonic sensor, or the like may be used to measure the distance from and the relative speed of the object. Alternatively, the plural sensors may be used to constitute a position and speed measuring device. The radar 22 corresponds to an example of the “travel environment information acquisition device” in the disclosure.

The navigation system 23 stores map information therein and can provide the map information to the controller 10. Based on the map information and current vehicle position information, the controller 10 identifies a road, the intersection, the traffic signal, a building, or the like that exists around (particularly, in the advancing direction of) the vehicle 1. The map information may be stored in the controller 10. The positioning system 24 is a GPS system and/or a gyroscopic system, and detects the position of the vehicle 1 (the current vehicle position information). Each of the navigation system 23 and the positioning system 24 also corresponds to an example of the “travel environment information acquisition device” in the disclosure.

The vehicle speed sensor 25 detects a speed of the vehicle 1 based on a rotational speed of the wheel or a driveshaft, for example. The acceleration sensor 26 detects acceleration of the vehicle 1. This acceleration includes acceleration in a front-rear direction of the vehicle 1 and acceleration in a lateral direction (i.e., lateral acceleration) thereof. In the present specification, the acceleration includes not only a change rate of the speed in a speed increasing direction but also a change rate of the speed in a speed reducing direction (i.e., deceleration).

The yaw rate sensor 27 detects a yaw rate of the vehicle 1. The steering angle sensor 28 detects a rotation angle (a steering angle) of a steering wheel of the steering device 5. The steering torque sensor 29 detects torque (steering torque) applied to a steering shaft via the steering wheel. The acceleration sensor 30 detects a depression amount of an accelerator pedal. The brake sensor 31 detects a depression amount of a brake pedal.

The in-vehicle camera 32 captures an image of the driver and outputs image data. The controller 10 detects the driver's sightline direction based on the image data received from the in-vehicle camera 32. The in-vehicle camera 32 corresponds to an example of the “sightline detector” in the disclosure.

The PCM 33 controls the drive power source 2 of the vehicle 1 to adjust the drive power of the vehicle 1. For example, the PCM 33 controls an ignition plug of the engine, a fuel injection valve, a throttle valve, a variable valve mechanism, the transmission 3, an inverter that supplies electric power to the electric motor, and the like. When the vehicle 1 has to be accelerated or decelerated, the controller 10 transmits the control signal to the PCM 33 so as to adjust the drive power.

The DSC 34 controls the drive power source 2 and the brake 4 of the vehicle 1 and thereby executes deceleration control and posture control of the vehicle 1. For example, the DSC 34 controls a hydraulic pump, a valve unit, and the like of the brake 4, and controls the drive power source 2 via the PCM 33. When it is necessary to execute the deceleration control or the posture control of the vehicle 1, the controller 10 transmits the control signal to the DSC 34 to adjust the drive power or generate the braking force.

The EPS 35 controls the steering device 5 of the vehicle 1. For example, the EPS 35 controls the electric motor, which applies the torque to the steering shaft of the steering device 5, and the like. When the advancing direction of the vehicle 1 has to be changed, the controller 10 transmits the control signal to the EPS 35 so as to change a steering direction.

The display 36 is provided in front of the driver in a cabin and displays the image information to the driver. As the display 36, for example, a liquid-crystal display or a head-up display is used. The speaker 37 is installed in the cabin and outputs various types of the voice information.

[Summary of Driver Abnormality Sign Detection]

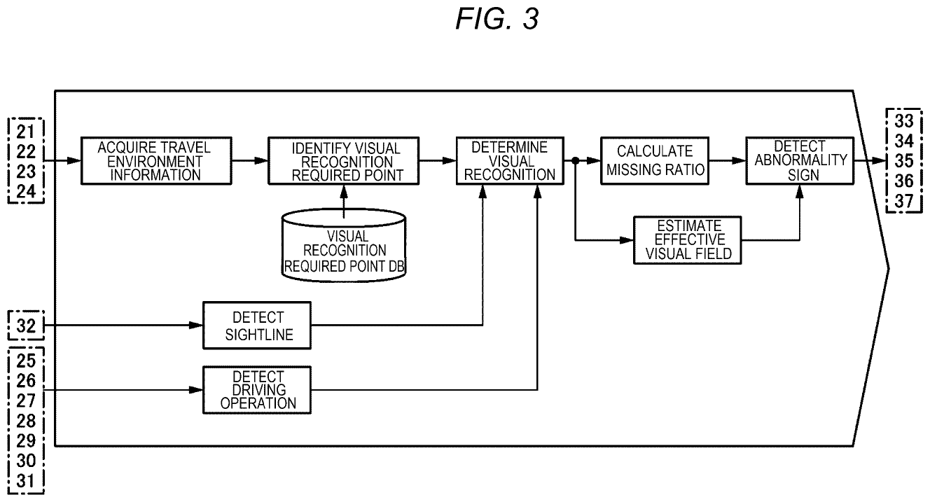

Next, a description will be made on a basic concept of driver abnormality sign detection executed by the above-described controller 10 in this embodiment with reference to FIG. 3. FIG. 3 is a control block diagram of the abnormality sign detection according to this embodiment.

In the abnormality sign state prior to an abnormal state where the driver has difficulty in driving, is the present inventors determined that the driver's attention function starts being declined due to a mild disease, aging, or the like before a full abnormal state is reached. In view of the above, the present inventors determined that the abnormality sign state could be detected by detecting this decline in the driver's attention function.

The driver's attention function mainly includes: a function to see plural objects simultaneously (a distributive attention function); a function to select the object from the plural objects and see the selected object (a selective attention function); a function to switch the object (a shifting attention function); and a function to keep seeing the object (a continuous attention function). As a result of research, the present inventors have discovered that, in the case where the attention function is declined due to a certain disease (a cardiac disorder, a brain disorder, hypoglycemia, or the like) or aging, the attention function is declined in an order of the distributive attention function—the selective attention function—the shifting attention function—the continuous attention function. That is, at an initial stage of the decline in the attention function, plural visual recognition required points that should be recognized visually (recognized by seeing with eyes) by the driver during driving can no longer be visually recognized at the same time, and the driver begins to miss some of the visual recognition required points due to the decline in the distributive attention function. Thus, the abnormality sign state can be detected based on severity of missing of the visual recognition required point.

As a result of the research, the present inventors have also discovered that, a patient whose attention function is declined has a narrow effective visual field (of the entire visual field, a range where information can be acquired effectively) and, thus, cannot visually recognize the object even when his/her sightline is directed to vicinity of the object. That is, at the initial stage of the decline in the attention function, since the effective visual field is narrowed, it becomes difficult to visually recognize the visual recognition required point, and consequently, the visual recognition required point is missed. Thus, the abnormality sign state can be detected by estimating a degree of narrowness of the effective visual field from a distance between the visual recognition required point and the sightline at the time when the visual recognition required point cannot be recognized visually.

Accordingly, the controller 10 in this embodiment is configured to identify the visual recognition required point based on the travel environment information, determines whether the driver has visually recognized the visual recognition required point based on the driver's sightline, and identifies the severity of missing of the visual recognition required point and a size of the effective visual field based on a fact that the driver has not visually recognized the visual recognition required point, so as to detect the driver's abnormality sign state.

More specifically, as illustrated in FIG. 3, the controller 10 acquires the travel environment information based on the signals received from the sensors including the outside camera 21, the radar 22, the navigation system 23, and the positioning system 24 (ACQUIRE TRAVEL ENVIRONMENT INFORMATION). The travel environment information is information on environment in which the vehicle 1 travels. Examples of the travel environment information are: type information and positional information of the object, such as the preceding vehicle, the parked vehicle, the pedestrian, the travel road, the lane marking (the lane divider, the white line, or the yellow line), the traffic signal, the traffic sign, the stop line, the intersection, or the obstacle, that exists around the vehicle 1; information on a type and a shape of the road on which the vehicle 1 travels; information on the peripheral building; and information on a current position of the vehicle 1.

Based on the acquired travel environment information, the controller 10 refers to a visual recognition required point database in which the travel environment and the visual recognition required points are stored in a mutually corresponding manner, and identifies the visual recognition required point to be visually recognized by the driver (IDENTIFY VISUAL RECOGNITION REQUIRED POINT). The visual recognition required point database is stored in the memory 10b or the like in advance.

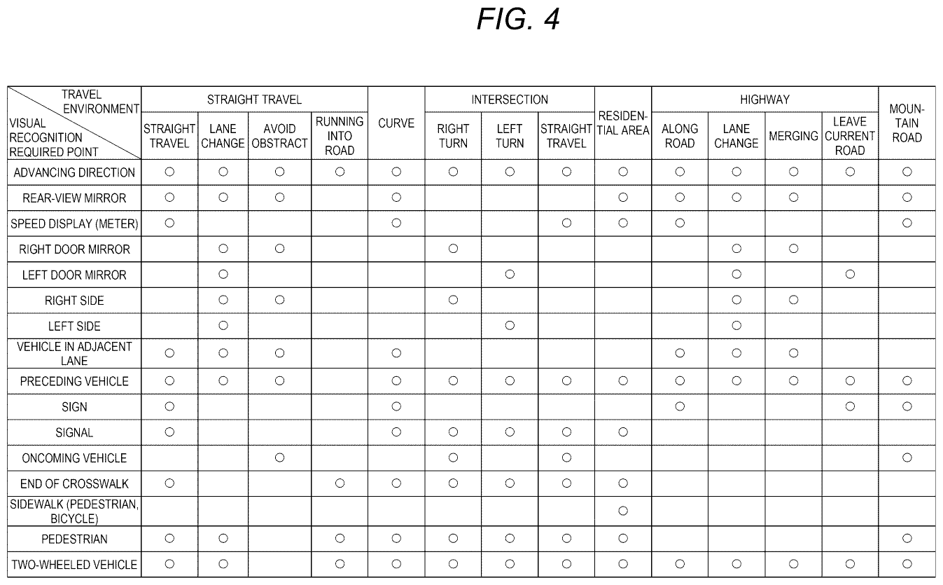

FIG. 4 is a table exemplifying information stored in the visual recognition required point database according to this embodiment. Each row in the visual recognition required point database exemplified in FIG. 4 represents the travel environment in which the vehicle 1 travels (“STRAIGHT TRAVEL” in “STRAIGHT TRAVEL”, “CURVE”, and the like). Meanwhile, each column in the visual recognition required point database exemplified in FIG. 4 represents the visual recognition required point to be visually recognized by the driver (“ADVANCING DIRECTION”, “REAR-VIEW MIRROR”, and the like). Then, the visual recognition required point to be visually recognized by the driver in each of the travel environments is circled. The controller 10 identifies the travel environment of the vehicle 1 based on the acquired travel environment information, and acquires the visual recognition required point in the identified travel environment from the visual recognition required point database. For example, in the case where the travel environment is “LEFT TURN” under “INTERSECTION”, in the example illustrated in FIG. 4, the visual recognition required points are identified as “ADVANCING DIRECTION”, “LEFT DOOR MIRROR”, “LEFT SIDE”, “PRECEDING VEHICLE”, “SIGNAL”, “END OF CROSSWALK”, “PEDESTRIAN”, and “TWO-WHEELED VEHICLE”.

The controller 10 detects the driver's sightline based on the signal received from the in-vehicle camera 32 (DETECT SIGHTLINE). Then, the controller 10 determines whether the driver has visually recognized the visual recognition required point based on the identified visual recognition required point and the detected driver's sightline (DETERMINE VISUAL RECOGNITION). More specifically, in the case where the driver's sightline is directed within a specified range including the visual recognition required point (for example, in the case where an angle defined by a direction from the driver's head toward the visual recognition required point and the driver's sightline falls within five degrees), the controller 10 determines that the driver has visually recognized the visual recognition required point. This specified range is set and stored in the memory 10b or the like in advance.

The controller 10 detects the driver's driving operation based on the signals received from the sensors including the vehicle speed sensor 25, the acceleration sensor 26, the yaw rate sensor 27, the steering angle sensor 28, the steering torque sensor 29, the acceleration sensor 30, and the brake sensor 31 (DETECT DRIVING OPERATION). Examples of the driving operation are an accelerating operation, a decelerating operation, a left turn operation, and a right turn operation.

Then, the controller 10 determines whether the driver has visually recognized the visual recognition required point based on the identified visual recognition required point as well as the detected driver's sightline and driving operation (DETERMINE VISUAL RECOGNITION). More specifically, in the case where the driver's sightline is directed within the specified range including the visual recognition required point (for example, in the case where the angle defined by the direction from the driver's head toward the visual recognition required point and the driver's sightline falls within 30 degrees), and the driving operation corresponding to the visual recognition required point is thereafter performed, the controller 10 determines that the driver has visually recognized the visual recognition required point. This specified range is set and stored in the memory 10b or the like in advance. Here, “driving operation corresponding to the visual recognition required point” means the driving operation to be performed by the driver when the driver visually recognizes the visual recognition required point (for example, in the case where the visual recognition required point is a red light, the driving operation corresponding thereto is the decelerating operation), is set in advance for each of the visual recognition required points, and is stored in the memory 10b or the like.

From a result of the visual recognition determination based on whether the driver's sightline is directed within the specified range including the visual recognition required point, the controller 10 calculates a ratio (a missing ratio) of the number of the visual recognition required points not visually recognized by the driver to the total number of the visual recognition required points (CALCULATE MISSING RATIO). In other words, a missing ratio Fr can be calculated by the following equation.

Missing ratio Fr=(the number of the visual recognition required points not visually recognized by the driver)/(the total number of the visual recognition required points)

Then, when the missing ratio Fr is equal to or higher than a specified threshold A (for example, 40% or higher), the driver is determined to be in the abnormality sign state, i.e., the driver's abnormality sign state is detected (DETECT ABNORMALITY SIGN). This threshold A is set and stored in the memory 10b or the like in advance. The threshold A may be set and stored based on the travel environment. For example, the threshold A may be set to be lower in a residential area than in a highway.

The controller 10 estimates the size of the effective visual field from the result of the visual recognition determination based on whether the driving operation corresponding to the visual recognition required point is performed when the driver's sightline is directed within the specified range including the visual recognition required point (ESTIMATE EFFECTIVE VISUAL FIELD). More specifically, the controller 10 acquires a minimum value (a minimum outside effective visual field angle) of the angles, each of which is defined by the direction from the driver's head toward the visual recognition required point and the driver's sightline, in regard to the visual recognition required points, each of which is determined not to be visually recognized by the driver. The controller 10 also acquires a maximum value (a maximum inside effective visual field angle) of the angles, each of which is defined by the direction from the driver's head toward the visual recognition required point and the driver's sightline, in regard to the visual recognition required points, each of which is determined to be visually recognized by the driver. Then, an intermediate value between the minimum outside effective visual field angle and the maximum inside effective visual field angle is defined as an effective visual field size (an effective viewing angle) Fv. In other words, the the effective visual field size Fv indicates how focused a driver is on the visual recognition required points.

Then, when the effective visual field size Fv is smaller than a specified threshold B (a central visual field size in which the drive can clearly see the object, e.g., smaller than five degrees), the driver is determined to be in the abnormality sign state. That is, the driver's abnormality sign state is detected (DETECT ABNORMALITY SIGN). This threshold B is set and stored in the memory 10b or the like in advance.

When detecting the abnormality sign state, the controller 10 transmits, to the PCM 33, the DSC 34, and the EPS 35, the control signal for appropriately actuating the drive power source 2, the transmission 3, the brake 4, and the steering device 5, and transmits, to the display 36 and the speaker 37, the control signal for outputting the desired information. For example, the controller 10 causes the display 36 to display the image information used to guide the driver's sightline to the visual recognition required point not visually recognized by the driver, and causes the speaker 37 to output the voice information.

[Abnormality Sign Detection Processing]

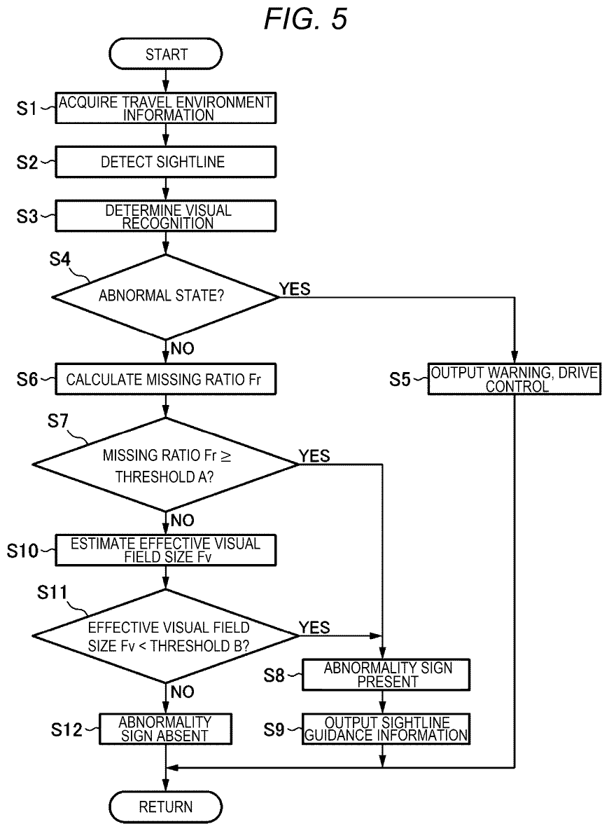

Next, a description will be made on a flow of detection processing of the abnormality sign state by the driver abnormality sign detection apparatus 100 in this embodiment with reference to FIG. 5. FIG. 5 is a flowchart of abnormality sign detection processing.

The abnormality sign detection processing in FIG. 5 is initiated when a power supply of the vehicle 1 is turned on, and is repeatedly executed by the controller 10 at a specified time interval (for example, every 0.05 to 0.2 second).

When the abnormality sign detection processing is initiated, first, the controller 10 acquires the travel environment information based on the signals received from the sensors including the outside camera 21, the radar 22, the navigation system 23, and the positioning system 24 (step S1).

In addition, the controller 10 detects the driver's sightline based on the signal received from the in-vehicle camera 32 (step S2). Then, the controller 10 identifies the visual recognition required point to be visually recognized by the driver based on the travel environment information acquired in step S1, and determines whether the driver has visually recognized the visual recognition required point based on the identified visual recognition required point and the driver's sightline detected in step S2 (step S3). As described above, in the case where the driver's sightline is directed within the specified range including the visual recognition required point, the controller 10 determines that the driver has visually recognized the visual recognition required point. In addition, in the case where the driver's sightline is directed within the specified range including the visual recognition required point, and thereafter the driving operation corresponding to the visual recognition required point is performed, the controller 10 determines that the driver has visually recognized the visual recognition required point.

Next, the controller 10 determines whether the driver is in the abnormal state based on the signals received from the sensors including the outside camera 21, the radar 22, the navigation system 23, the positioning system 24, the vehicle speed sensor 25, the acceleration sensor 26, the yaw rate sensor 27, the steering angle sensor 28, the steering torque sensor 29, the acceleration sensor 30, the brake sensor 31, and the in-vehicle camera 32 (step S4).

For example, as in the technique disclosed in JP-A-2021-077136, the controller 10 detects an amplitude and a frequency of the vehicle driver's saccade, detects such an attention level that is increased as the number of caution points to be checked by the driver during the travel is increased in the external environment of the vehicle, and determines whether the driver is in the abnormal state based on the attention level and the amplitude and the frequency of the driver's saccade. Alternatively, the controller 10 detects the driver's sightline direction, a posture (a position of an upper body or the head), opening amounts of the driver's eye lids, the driver's steering wheel grip force, or the like. In this way, based on such a detection result, the controller 10 can also determine that the driver is in the abnormal state. For example, in the case where stability of the sightline direction is lower than a specified value, in the case where stability of the posture is lower than a specified value, in the case where the eye lids keep closed for a specified time or longer, or in the case where the steering wheel grip force is smaller than a specified value, the controller 10 can determine that the driver is in the abnormal state. In addition, for example, in the case where stability of the position of the vehicle 1 from a center line on the travel road, stability of the steering angle, or the like is lower than a specified value, the controller 10 can estimate that the driver is in the abnormal state.

As a result, if it is determined that the driver is in the abnormal state (step S4: YES), the controller 10 transmits the control signal to the display 36 and the speaker 37 and causes the display 36 and the speaker 37 to output a warning. The controller 10 also transmits, to the PCM 33, the DSC 34, and the EPS 35, the control signal to appropriately actuate the drive power source 2, the transmission 3, the brake 4, and the steering device 5, and thereby controls the behavior of the vehicle 1 such that the vehicle 1 is safely stopped on a road shoulder, for example (step S5). After step S5, the controller 10 terminates the abnormality sign detection processing.

On the other hand, if it is determined that the driver is not in the abnormal state (step S4: NO), the controller 10 calculates the missing ratio Fr from the result of the visual recognition determination based on whether the driver's sightline is directed within the specified range including the visual recognition required point (step S6).

Next, the controller 10 determines whether the missing ratio Fr calculated in step S6 is equal to or higher than the specified threshold A (step S7). As a result, if the missing ratio Fr is equal to or higher than the specified threshold A (step S7: YES), the controller 10 determines that the driver is in the abnormality sign state. That is, the driver's abnormality sign state is detected (step S8).

Next, the controller 10 transmits the control signal to the display 36 and the speaker 37, and causes the display 36 and the speaker 37 to respectively output the image information and the voice information (sightline guidance information) used to guide the driver's sightline to the visual recognition required point not visually recognized by the driver (step S9). After step S9, the controller 10 terminates the abnormality sign detection processing.

On the other hand, in step S7, if the missing ratio Fr is not equal to or higher than the specified threshold A (is lower than the threshold A) (step S7: NO), the controller 10 estimates the effective visual field size Fv from the result of the visual recognition determination in step S3 based on whether the driving operation corresponding to the visual recognition required point is performed when the driver's sightline is directed within the specified range including the visual recognition required point (step S10).

Next, the controller 10 determines whether the effective visual field size Fv estimated in step S10 is smaller than the specified threshold B (step S11). As a result, if the effective visual field size Fv is smaller than the specified threshold B (step S11: YES), the controller 10 determines that the driver is in the abnormality sign state. That is, the driver's abnormality sign state is detected (step S8). In this case, the controller 10 causes the display 36 and the speaker 37 to output the sightline guidance information (step S9), and terminates the abnormality sign detection processing.

On the other hand, in step S11, if the effective visual field size Fv is not smaller than the specified threshold B (is equal to or larger than the threshold B) (step S11: NO), the controller 10 determines that the driver is not in the abnormality sign state. That is, the driver's abnormality sign state is not detected (step S12). After step S12, the controller 10 terminates the abnormality sign detection processing.

In the abnormality sign detection processing in this embodiment, the controller 10 detects the driver's abnormality sign state based on each of whether the missing ratio Fr is equal to or higher than the specified threshold A and whether the effective visual field size Fv is smaller than the specified threshold B. However, the controller 10 may detect the abnormality sign state based on only one of the missing ratio Fr and the effective visual field size Fv.

In addition, in the abnormality sign detection processing in this embodiment, the controller 10 detects the driver's abnormality sign state when determining in step S4 that the driver is not in the abnormal state (step S4: NO). However, the determination on whether the driver is in the abnormal state may not be made, or the determination on whether the driver is in the abnormal state may be made after the abnormality sign state is detected.

Furthermore, in this embodiment, the effective visual field size Fv is estimated from the result of the visual recognition determination based on whether the driving operation corresponding to the visual recognition required point is performed when the driver's sightline is directed within the specified range including the visual recognition required point. However, the effective visual field size may be estimated by a different method therefrom. For example, the effective visual field size can also be estimated based on an angle defined by a direction from the driver's head toward the object with high saliency (for example, the object with a significant color difference or luminance difference from the surrounding area, or the object with large motion with respective to the surroundings) and the driver's sightline at the time when the driver's sightline is directed to such an object.

[Operational Effects]

Next, a description will be made on operational effects of the driver abnormality sign detection apparatus 100 in the above-described embodiment.

The controller 10 identifies the visual recognition required point to be visually recognized by the driver based on the travel environment information, determines whether the driver has visually recognized the visual recognition required point based on the driver's sightline, and detects the driver's abnormality sign state based on the fact that the driver has not visually recognized the visual recognition required point. Thus, the abnormality sign state may be detected by using the fact that the driver has not been able to visually recognize the visual recognition required point due to the decline in the distributive attention function or a reduction in the effective visual field at the initial stage of the decline in the driver's attention function. In this way, the driver's abnormality sign state may be detected at a stage at which no obvious difference appears to the amplitude and the frequency of the driver's saccade and which is sufficiently earlier than onset of the abnormal state where the driver has difficulty in driving, for example.

The controller 10 determines that the driver has visually recognized the visual recognition required point in the case where the driver's sightline is directed within the specified range including the visual recognition required point, and detects the driver's abnormality sign state based on the number of the visual recognition required points not visually recognized by the driver. Accordingly, when the driver can no longer visually recognize the plural visual recognition required points at the same time due to the decline in the distributive attention function and starts missing the visual recognition required points at the initial stage of the decline in the attention function, the abnormality sign state can be detected based on the severity of missing of the visual recognition required point. In this way, the driver's abnormality sign state at the stage sufficiently earlier than onset of the abnormal state where the driver has difficulty in driving may be appropriately detected.

The controller 10 is configured to detect the driver's abnormality sign state in the case where the missing ratio of the visual recognition required point is equal to or higher than the specified value. Accordingly, when the driver can no longer visually recognize the plural visual recognition required points at the same time due to the decline in the distributive attention function and the missing ratio of the visual recognition required point becomes equal to or higher than the specified value at the initial stage of the decline in the attention function, the abnormality sign state can be detected. In this way, the driver's abnormality sign state at the stage sufficiently earlier than the onset of the abnormal state where the driver has difficulty in driving may be appropriately detected.

The controller 10 estimates the effective visual field size that can be visually recognized by the driver based on the distance between the driver's sightline and the visual recognition required point not visually recognized by the driver. Then, in the case where the estimated effective visual field size is smaller than the specified value, the driver's abnormality sign state is detected. Accordingly, when the effective visual field is narrowed and the driver starts missing the visual recognition required point at the initial stage of the decline in the attention function, the abnormality sign state can be detected by estimating the degree of the narrowness of the effective visual field. In this way, the driver's abnormality sign state may be appropriately detected at the stage sufficiently earlier than the onset of the abnormal state where the driver has difficulty in driving.

The driver abnormality sign detection apparatus 100 further includes the display 36 and the speaker 37, each of which outputs the information to the driver. When detecting the driver's abnormality sign state, the controller 10 causes the display 36 and the speaker 37 to output the information to guide the driver's sightline to the visual recognition required point. Accordingly, in the case where the driver's abnormality sign state is detected, the driver's sightline can be guided to the visual recognition required point. Therefore, the appropriate driver assistance may be provided to the driver who starts missing the visual recognition required point at the initial stage of the decline in the attention function.

No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

The present disclosure is not limited to only the above-described embodiments, which are merely exemplary. It will be appreciated by those skilled in the art that the disclosed systems and/or methods can be embodied in other specific forms without departing from the spirit of the disclosure or essential characteristics thereof. The presently disclosed embodiments are therefore considered to be illustrative and not restrictive. The disclosure is not exhaustive and should not be interpreted as limiting the claimed invention to the specific disclosed embodiments. In view of the present disclosure, one of skill in the art will understand that modifications and variations are possible in light of the above teachings or may be acquired from practicing of the disclosure. The scope of the invention is indicated by the appended claims, rather than the foregoing description.

### DESCRIPTION OF REFERENCE SIGNS AND NUMERALS

1 Vehicle
10 Controller
100 Driver abnormality sign detection apparatus
21 Outside camera
22 Radar
23 Navigation system
24 Positioning system
25 Vehicle speed sensor
26 Acceleration sensor
27 Yaw rate sensor
28 Steering angle sensor
29 Steering torque sensor
30 Acceleration sensor
31 Brake sensor
32 In-vehicle camera
36 Display
37 Speaker

1. A driver pre-abnormal detection apparatus that detects a pre-abnormal state of a driver who drives a vehicle, the driver pre-abnormal detection apparatus comprising:
a travel environment information acquisition sensor that acquires travel environment information of the vehicle;
a sightline detector that detects the driver's sightline; and
a controller configured to detect the driver's pre-abnormal state based on the travel environment information and the driver's sightline, wherein
the controller is configured to:
identify a visual recognition required point to be visually recognized by the driver based on the travel environment information;
determine whether the driver has visually recognized the visual recognition required point based on the driver's sightline; and
detect the driver's pre-abnormal state based on a fact that the driver has not visually recognized the visual recognition required point.

2. The driver pre-abnormal detection apparatus according to claim 1, wherein
the controller is configured to:
determine that the driver has visually recognized the visual recognition required point in the case where the driver's sightline is directed within a specified range including the visual recognition required point; and
detect the driver's pre-abnormal state based on the number of the visual recognition required point not visually recognized by the driver.

3. The driver pre-abnormal detection apparatus according to claim 2, wherein
the controller is configured to detect the driver's pre-abnormal state in the case where a ratio of the number of the visual recognition required point not visually recognized by the driver to the total number of the visual recognition required points is equal to or larger than a specified value.

4. The driver pre-abnormal detection apparatus according to claim 3, further comprising:
an information output device that outputs information to the driver, wherein
the controller is configured to cause the information output device to output information used to guide the driver's sightline to the visual recognition required point in the case where the driver's pre-abnormal state is detected.

5. The driver pre-abnormal detection apparatus according to claim 2, further comprising:
an information output device that outputs information to the driver, wherein
the controller is configured to cause the information output device to output information used to guide the driver's sightline to the visual recognition required point in the case where the driver's pre-abnormal state is detected.

6. The driver pre-abnormal detection apparatus according to claim 1, wherein
the controller is configured to:
estimate a size of an effective visual field visually recognized by the driver based on a distance between the driver's sightline and the visual recognition required point not visually recognized by the driver; and
detect the driver's pre-abnormal state in the case where the estimated effective visual field size is smaller than a specified value.

7. The driver pre-abnormal detection apparatus according to claim 6, further comprising:
an information output device that outputs information to the driver, wherein
the controller is configured to cause the information output device to output information used to guide the driver's sightline to the visual recognition required point in the case where the driver's pre-abnormal state is detected.

8. The driver pre-abnormal detection apparatus according to claim 1, further comprising:
an information output device that outputs information to the driver, wherein
the controller is configured to cause the information output device to output information used to guide the driver's sightline to the visual recognition required point in the case where the driver's pre-abnormal state is detected.

9. A driver pre-abnormal detection circuit for detecting a pre-abnormal state of a driver who drives a vehicle, the driver pre-abnormal detection circuit being configured to:
acquire travel environment information of the vehicle;
acquire the driver's sightline;
identify a visual recognition required point to be visually recognized by the driver based on the travel environment information;
determine whether the driver has visually recognized the visual recognition required point based on the driver's sightline; and
detect the driver's pre-abnormal state based on a fact that the driver has not visually recognized the visual recognition required point.

10. The driver pre-abnormal detection circuit according to claim 9, wherein the driver pre-abnormal detection circuit is configured to:
determine that the driver has visually recognized the visual recognition required point in the case where the driver's sightline is directed within a specified range including the visual recognition required point; and
detect the driver's pre-abnormal state based on the number of the visual recognition required point not visually recognized by the driver.

11. The driver pre-abnormal detection circuit according to claim 10, wherein the driver pre-abnormal detection circuit is configured to detect the driver's pre-abnormal state in the case where a ratio of the number of the visual recognition required point not visually recognized by the driver to the total number of the visual recognition required points is equal to or larger than a specified value.

12. The driver pre-abnormal detection circuit according to claim 9, wherein the driver pre-abnormal detection circuit is configured to output information used to guide the driver's sightline to the visual recognition required point in the case where the driver's pre-abnormal state is detected.

13. The driver pre-abnormal detection circuit according to claim 9, wherein the driver pre-abnormal detection circuit is configured to:
estimate a size of an effective visual field visually recognized by the driver based on a distance between the driver's sightline and the visual recognition required point not visually recognized by the driver; and
detect the driver's pre-abnormal state in the case where the estimated effective visual field size is smaller than a specified value.

14. A non-transitory computer readable storage device having computer readable instructions that when executed by circuitry cause the circuitry to:
acquire travel environment information of a vehicle being driven by a driver;
acquire the driver's sightline;
identify a visual recognition required point to be visually recognized by the driver based on the travel environment information;
determine whether the driver has visually recognized the visual recognition required point based on the driver's sightline; and
detect a pre-abnormal state of the driver based on a fact that the driver has not visually recognized the visual recognition required point.
